(19) United States
(12) Patent Application Publication
(10) Pub. No.: US 2025/0242087 A1
(43) Pub. Date: Jul. 31, 2025

(54) DEVICES AND METHODS FOR IMPROVING THE GROWTH OF HOST TISSUE INTO AN ALLOGRAFT

(71) Applicant: Lipocosm, LLC, Key Biscayne, FL (US)

(72) Inventor: Roger K. Khouri, Key Biscayne, FL (US)

(73) Assignee: Lipocosm, LLC, Key Biscayne, FL (US)

(21) Appl. No.: 19/080,088

(22) Filed: Mar. 14, 2025

Related U.S. Application Data
(63) Continuation of application No. 17/719,456, filed on Apr. 13, 2022, now abandoned.
(60) Provisional application No. 63/175,660, filed on Apr. 16, 2021.

(51) Int. Cl. A61L 27/54

(52) U.S. Cl. CPC A61L 27/54; A61L 2300/414; A61L 2300/64

(57) ABSTRACT

Methods and devices that accelerate and improve graft induced growth of host living cells by application of mechanical tension over the graft/host construct that invites host cells to grow, multiply, and repopulate the graft matrix.

### CROSS-REFERENCE TO RELATED APPLICATIONS

This application claims priority to provisional application Ser. No. 63/175,660, filed Apr. 16, 2021, the entire contents of which are incorporated herein by reference.

### FIELD OF THE INVENTION

This invention relates to methods and devices that accelerate and improve the growth of host living cells into materials derived from an allograft or xenograft.

### BACKGROUND

Grafts in general may provide useful replacement for diseased or lost human tissues and organs. Unfortunately, grafts are generally fraught with failure due to the host immune system rejecting the foreign tissue. One alternative around this limitation has been to deplete the graft of its immunologic living cells and leave behind a decelullarized matrix that is typically generic across species and not immunogenic. However, to survive and function, this decellularized graft matrix needs to promote the growth of the recipient tissue and invite the ingrowth of host tissue that would re-populate it, a process that is slow, quite limited, and unpredictable. Current attempts with decellularized grafts have therefore met very limited success.

It would be beneficial to provide a method to accelerate the growth of the host tissues in response to the graft material and to improve its ingrowth into the graft.

Additionally, research has been conducted into an alternative to using human material for grafting on human patients. Xenografts from animals such as pigs can provide one possible grafting material. Thus, it would also be beneficial to provide a method to accelerate ingrowth of host tissue into specially treated xenografts.

### SUMMARY OF THE INVENTION

The present invention provides methods and devices for accelerating ingrowth of host tissue into grafts and for increasing the growth promoting effect of materials derived from grafts.

The present invention expands the scope of external mechanical tension and applies it to accelerate and improve the growth of allografts, xenografts, and the growth promoting effects of materials derived from such grafts.

In accordance with one aspect of the present invention, a method of accelerating ingrowth of host living cells into a graft is provided comprising applying a mechanical tension over an interface of the graft and host cells.

In some embodiments, the mechanical tension is applied to invite the host cells to exit the host and migrate inside the graft. In some embodiments, the mechanical tension is applied by an external expansion device which is placed over the interface and applies a vacuum to the region of the graft matrix. In some embodiments, mechanical tension is applied by an external device which is placed over the interface and passively prevents intrinsic recoil of pre-stretched tissue.

In some embodiments, the graft is a decellularized allograft; in other embodiments, the graft is a decellularized xenograft. The decellularized graft material is a living-cell-free protein matrix derived from the graft that has retained the biologic signals and clues that induce the growth and differentiation of host tissue. This matrix might have the fibrous structural framework of the original tissue preserved, in which case the graft is a structure in itself; or the fibrous structure might be disrupted, in which case the graft is a paste or an injectable fluid. In some further embodiments, the graft is a decellularized matrix and the method further comprises infusing the graft matrix with one or more materials that invite tissue ingrowth. The one or more materials can be infused prior to graft application and/or during graft application.

In some embodiments, mechanical tension is applied by a constant suction at a sufficient low level so as not to collapse capillary walls and block blood flow. In other embodiments, mechanical tension is applied by intermittent suction to provide relaxation periods to temporarily restore circulation.

In accordance with another aspect of the present invention, a method of accelerating chimerism of an organ or tissue transplant is provided comprising applying mechanical tension over an interface of a graft and host cells. In some embodiments, the graft is a decelullarized allograft; in other embodiments, the graft is a decellularized xenograft.

In accordance with another aspect of the present invention, a method of augmenting soft tissue is provided comprising injecting material derived from fat into a region of a body of a patient expanded by application of a distractive force to increase the volume of the region of the body. In some embodiments, the graft is derived from an allograft; in other embodiments, the graft is derived from a xenograft.

In accordance with another aspect of the present invention, a method of enlarging soft tissue is provided comprising injecting graft material derived from homologous tissue into a region of a body of a patient expanded by application of a force, e.g., a distractive force, to mechanically expand that tissue. The distractive force induces the recipient site to generate a tissue engineering scaffold. In this invention, the scaffold is seeded not with living autograft, but with allografts or xenografts that have been processed to lose their immunogenicity and maintain their particular growth and differentiation factors and clues.

This might apply to enlarging/regenerating a deficient liver by injecting material derived from liver grafts, or regenerating a deficient kidney by inserting material derived from kidney grafts. While the inventor contemplates living allografts and xenografts, in most current applications the grafts are preserved, cell free, mostly proteinaceous material that might contain other native biologic compounds such as lipids, glycoproteins, peptides, polysaccharides, and amino acids.

A practical application of this invention is to increase the volume of a fatty body region such as the breast by injecting it with material derived from fat not necessarily coming from the patient. In some embodiments, the injected material is derived from an animal; in other embodiments, the injected material is derived from a human.

Another practical application of this invention is to reconstruct/regenerate the human nipple in women who lost their breast(s) from cancer resections. By applying external mechanical tension to generate a recipient scaffold and injecting that scaffold with autologous fat grafts, the current inventor has been able to successfully restore/regenerate a living breast mound after mutilating total mastectomies. However, giving patients back a nipple to complete their reconstruction has remained a challenge. Despite multiple attempts, allograft nipples have not succeeded. Live allografts are highly immunogenic and rejected, and decellularized allografts while less immunogenic, have limited ability to induce the ingrowth of host tissue to resurrect them. The current invention provides in some embodiments adding external mechanical tension to accelerate the ingrowth of recipient tissue into material derived from allograft nipples and that by themselves have limited capacity of success.

Note that in the above applications, and other clinical applications, the present invention provides adding external mechanical tension to accelerate, improve and augment the limited success of allografts and xenografts whether live or decellularized, whether still in their original structural form and shape or whether their structural framework is lost and they are in a paste or fluid injectable form.

In some embodiments, the distractive force is applied by a vacuum; in other embodiments it is applied by a passive device that prevents the intrinsic recoil of stretched/expanded tissue.

### BRIEF DESCRIPTION OF DRAWINGS

Preferred embodiments of the present invention described in detail hereinbelow with reference to the drawings, wherein:

FIG. 1 is a schematic representation of a dome utilizing vacuum for augmenting tissue.

FIG. 2 is a schematic representation of a rigidifying brace for maintaining distended tissue.

FIG. 3 is a schematic representation of a dome utilizing vacuum to apply mechanical tension over an acellular nipple allograft matrix grafted over a surgically created wound recipient defect in a breast mound missing a nipple.

FIG. 4 is a schematic representation of a dome utilizing vacuum to apply mechanical tension over an entire breast induced to grow by insertion of an allograft matrix.

FIG. 5 is a schematic representation of a dome utilizing vacuum to apply mechanical tension over deficient soft tissue dome that needs to be enlarged. The prepared graft matrix is injected as a bolus and combined with the effect of tension, host cells grow and migrate to replace the inert injected matrix with living tissue.

FIG. 6 is a schematic representation of a dome utilizing vacuum to apply mechanical tension over a deficient soft tissue that needs to be enlarged. The graft matrix in liquid form is diffusely injected into the recipient. Expansion potentiates the growth stimulating effect of the graft matrix as a result the recipient site grows and recipient cells divide and migrate into the grafted sites.

### DESCRIPTION OF PREFERRED EMBODIMENTS

A method for retaining an organ in an expanded state, for utilization with the present invention involves in some embodiments, applying at least one force to the organ to place the organ in the expanded state. For example, the at least one force may be applied using injection, a vacuum, surface tension, or other internal or external force that induces distention in the organ. In some embodiments, the Brava Bra (see, for example, U.S. Pat. Nos. 5,536,233; 5,662,583; 5,676,634; 5,695,445; 5,701,917; 6,478,656; 6,500,112; 6,641,527; and 6,699,176, all of which are incorporated herein by reference in their entirety) and/or an external passive expander splint (see, for example, U.S. Pat. Nos. 9,066,795 and 9,522,058, also incorporated herein by reference herein in their entirety) may induce the distractive force and, over a period of time, place the organ in the expanded state.

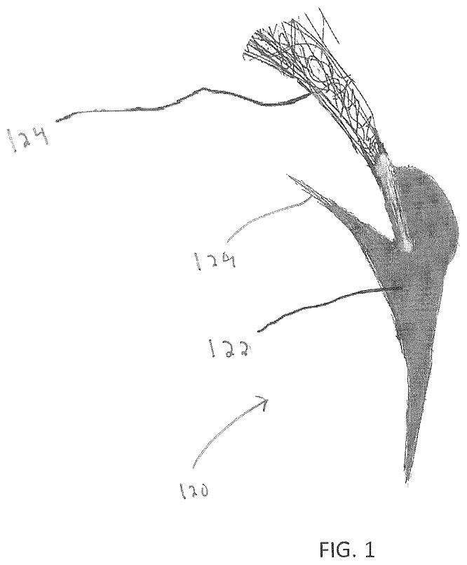

Commonly assigned co-pending application Ser. No. 17/534,527, filed Nov. 24, 2021 (the entire contents of which are incorporated herein by reference) discloses improvements to prior vacuum expanders (tissue expanders) by effectively 1) preserving the vacuum by preventing air leaks; 2) balancing the distractive force applied to the breast with the counter-force exerted by the rim of the external vacuum expander in contact with the surrounding skin to prevent excessive pressure that would collapse capillary circulation and lead to pressure ulcerations; and 3) reducing the shear stresses that develops at the junction between the tensed skin inside the vacuum shell with the skin firmly held down and anchored by the inner lip of the rim to reduce skin irritation, blistering and ulceration which is caused by excess shear forces concentrated at the inner lip of the rim. FIG. 1, corresponding to FIG. 8 of the 17/534,527 application, illustrates an embodiment having the foregoing improvements as disclosed in the 17/534,527 application. Dome shell 124 is attached to the rim 122 of expander 120 and has an additional inner thin and compliant deflecting rim 122 extending inside the dome aperture that increases the contact area against the skin to improve the vacuum seal. The rim includes a feathered region 124′. More details of the vacuum expander, as well as alternative expanders, are disclosed in the above listed applications, incorporated by reference herein in their entirety.

Note some of the devices in the patents are utilized with fat grafting and stabilize the graft as the fat fills the void. U.S. Pat. No. 8,066,691, incorporated herein by reference in its entirety, discloses some methods for injecting graft material.

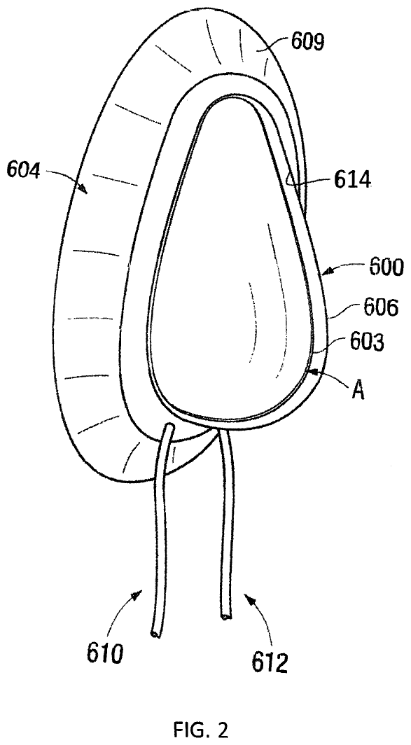

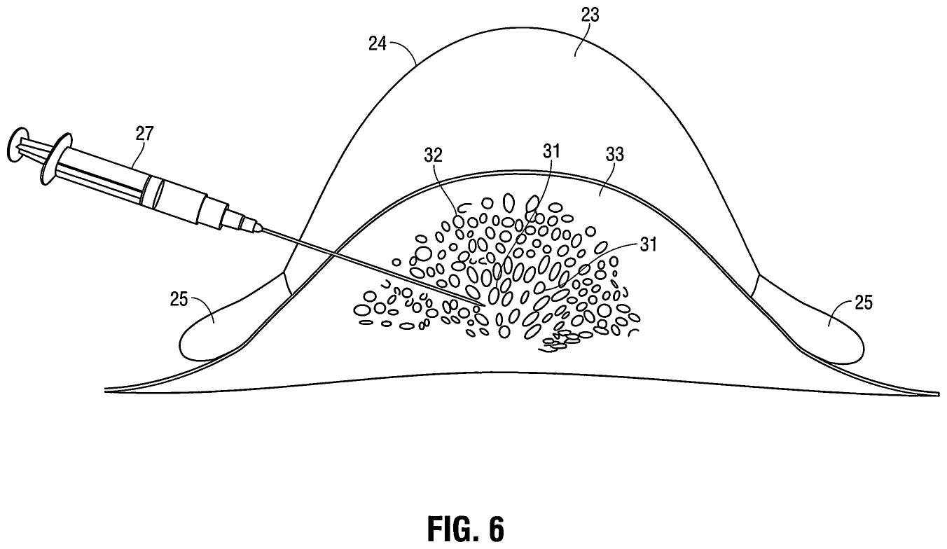

Commonly assigned co-pending application Ser. No. 17/268,302, filed Feb. 12, 2021 (the entire contents of which are incorporated herein by reference) provides a rigidifying brace to maintain distended tissue. Commonly assigned provisional application 63/175,611, filed Apr. 16, 2021 and utility application entitled “Rigidifying Brace”, Ser. No. 17/719,446 filed on Apr. 13, 2022 (the entire contents of both applications are incorporated herein by reference) also disclose a rigidifying brace to maintain the distention. FIG. 2, corresponding to FIG. 6A of the provisional application 63/175,611, illustrates an embodiment of the passive rigidifying brace. The brace 600 has a middle layer with a plurality of components, e.g., soft paper, in which a vacuum is applied to cause the plurality of components to interlock and/or compress and/or rigidify. The middle layer is between outer layer 606 and inner layer 603. The rigidifying braces can be utilized after grafting. The brace 600 can be utilized for placement over a breast (or other organs/tissue) and has a thin sheet 604 which is placed over a section of the outer layer 606 and extends beyond a periphery of the outer layer 606. The sheet has an inner surface 608 with an adhesive surface covered by a peel away strip. Ports 610, 612 are utilized to apply vacuum to the middle layer and to the region between the inner layer and skin. More details of the rigidifying brace, and alternative rigidifying braces, are disclosed in the above listed rigidifying brace applications, incorporated by reference herein in their entirety.

The present invention provides in some embodiments methods and devices that accelerate and improve the ingrowth of host living cells into a decellularized graft. The decellularized graft is formed by depletion of its immunologic living cells which leave behind a decellularized protein matrix. Mechanical tension induces tissue growth and vacuum applied over a wound accelerates its closure. Thus, the judicious application of mechanical tension over a foreign graft/host interface, whether through vacuum or otherwise, invites host cells to exit the host and migrate and grow inside the graft, repopulate it, and turn it into a living autologous transplant.

The present invention provides in some embodiments methods and devices that accelerate and improve the ingrowth of host living cells into a xenograft or an allograft by the judicious application of mechanical tension over the graft/host interface, whether through vacuum or otherwise, which invite host cells to exit the host and migrate and grow inside the treated decellularized graft, repopulate it, and turn it into a living autologous transplant.

Thus, as can be appreciated, such grafting can include allografts or xenografts, preferably after depletion of the immunologic living cells and other known immunogenic components. The graft can be composed of cell free material derived from fat (human or animal, e.g., bovine or pig) and the treated graft could then either have retained its original fibrous structural framework and thus manipulated as a structural graft, or its treatment could have made it lose its structural shape and turned into a paste or an injectable form. This provides a method of augmenting tissue comprising injecting this material derived from fat into a region of the body expanded by a force, e.g., a distractive force, to increase the volume of the body region. The foregoing vacuum expanders can apply the distractive force. The foregoing rigidifying braces can retain the tissue in the augmented state and subject them to a distractive force by counteracting their recoil.

External expansion devices can be utilized to apply tension. In some embodiments, the expansion is induced by active vacuum and in other embodiments by passively preventing the intrinsic recoil forces of pre-stretched tissues. If the graft needs to be an external surface tissue material such as a nipple, the expansion devices are preferably applied sterile over the sterile graft matrix—open recipient tissue interface construct. Tension applied upon the living recipient fibrovascular scaffold would then induce capillaries from the host to migrate and grow into the allograft matrix. If the graft is in a form that can be injected or inserted inside the recipient, the graft material needs to be sterile, but since the external expansion device interfaces with intact recipient skin, the expansion device does not necessarily need to be sterile.

Tension can be applied through constant suction, at a sufficiently low level so as not to collapse capillary walls and block blood flow, or, alternatively at higher levels which can be more effective, yet applied at non-traumatic levels, and applied intermittently for short periods of time followed by relaxation periods to temporary restore circulation. A specially designed sponge or other biocompatible material can be included inside the expansion device as an interfacing medium between the allograft and the inner surface of the expansion device. Tension applied through vacuum or otherwise would invite not only cells but also fluid rich in cytokines and other growth promoting factors to exit the open graft-recipient interface and permeate the interfacing medium and the allograft matrix and provide a biologically favorable environment for tissue ingrowth.

Another useful addition and/or alternative to accelerate the process of tissue migration into the graft would be to infuse the graft matrix with materials that invite ingrowth. This infusion could be done prior to graft application or concurrent with it. The interfacing medium could also be similarly infused. Examples of such factors might include Platelet Rich Plasma, liposuctioned host tissue or material fractions derived from that liposuction, stem cells, cytokines, growth factors, or other bioactive growth promoting products.

Furthermore, it is also well established that standard living allografts survive better when cells in the graft are gradually replaced/repopulated by host tissue, a process called chimerism. The higher degree of chimerism the less immunogenic and therefore less rejection prone the allograft becomes. Therefore, by accelerating the process of host tissue migration into an allograft, this invention can also be extended to encompass a method to accelerate/improve the chimerism of organ and tissue transplants. This would apply to skin allografts, fat allografts and composite allotransplantation such as face and hand transplants; and also, potentially to solid organ transplants.

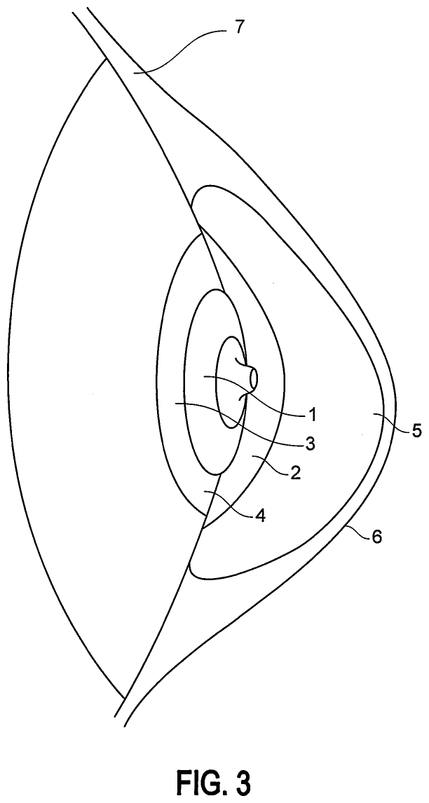

FIGS. 3-6 illustrate examples of use of the present invention. The domes utilized can be those disclosed in the commonly assigned patents and patent applications referenced above.

FIG. 3 is a schematic representation of a dome utilizing vacuum to apply mechanical tension over an acellular nipple allograft matrix grafted over a surgically created wound recipient defect in a breast mound missing a nipple. As shown, reference numeral 1 designates the nipple and aerola allograft with a sterile protective biologic dressing 2 placed thereover. The protective dressing acts as a stent to immobilize the graft while preserving/absorbing the growth promoting substances leached from the graft and the recipient and still transmitting the active expansion force. The wound recipient site is designated by reference numeral 3 and the intact surrounding skin by reference numeral 4. A vacuum expander dome 6 is placed over the breast creating a vacuum expansion space 5. The rim 7 of the expander dome 6 is in sealing contact with the skin. As an alternative, the inner surface of the expander is directly in contact with the protective dressing bypassing the need for a vacuum expansion space.

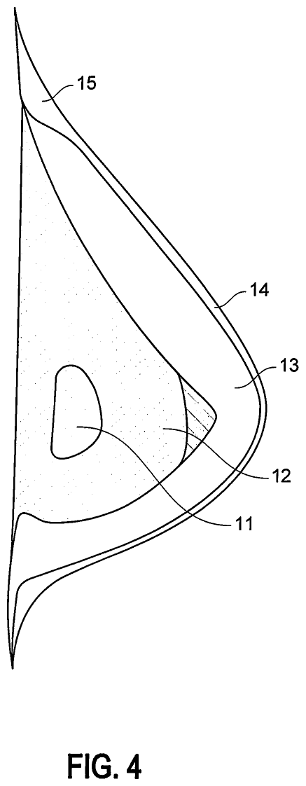

FIG. 4 is a schematic representation of a dome 14 utilizing vacuum to apply mechanical tension over an entire breast induced to grow by insertion of an allogenic or xenogenic graft matrix or graft matrix paste or liquid bolus 11. Dome 14 is placed over the breast recipient site 12 and the dome 14 via vacuum forms a vacuum expansion space 13 sealed by the skin sealing contact of expander rim 15.

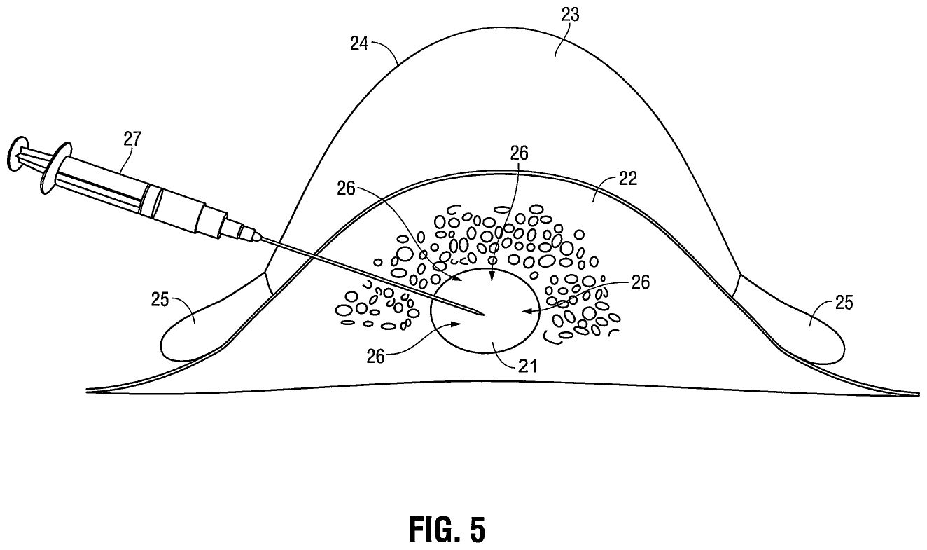

FIG. 5 is a schematic representation of a dome utilizing vacuum to apply mechanical tension over deficient soft tissue that needs to be enlarged. The prepared allogenic or xenogenic graft matrix is inserted (e.g., injected) by injection device 27 as a fluid graft bolus (or surgically inserted as a graft matrix paste or a biologic scaffold containing the graft fluid) and combined with the effect of tension, host cells grow and migrate to replace the inert inserted matrix with living tissue. The expander dome 24, with sealing rim 25 in sealing contact with the skin, forms vacuum expansion space 23. The injected material is designated by reference numeral 21, the soft tissue recipient site is designated by reference numeral 22 and the recipient cells stimulated to grow and migrate into the graft is designated by reference numeral 26.

FIG. 6 is a schematic representation of a dome utilizing vacuum to apply mechanical tension over a deficient soft tissue that needs to be enlarged. The graft matrix in liquid form is diffusely injected by injection device 27 into the recipient. Expansion potentiates the growth stimulating effect of the graft matrix as a result the recipient site grows and recipient cells divide and migrate into the grafted sites. The expander dome 24, with sealing rim 25 in sealing contact with the skin, forms vacuum expansion space 23. The injected graft material droplets are designated by reference numeral 31, the recipient cells growing and migrating into the grafts are designated by reference numeral 32 and the soft tissue recipient site is designated by reference numeral 33.

The foregoing description has been presented for purposes of illustration. It is intended that the specification and examples be considered as exemplary only. It is not exhaustive and is not limited to precise forms or embodiments disclosed. Other embodiments will be apparent to those skilled in the art from consideration of the specification and practice of the embodiments disclosed herein. Modifications and adaptations of the embodiments will be apparent from consideration of the specification and practice of the disclosed embodiments.

The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps and/or inserting or deleting steps.

The features and advantages of the disclosure are apparent from the detailed specification, and thus, it is intended that the appended claims cover all systems and methods falling within the true spirit and scope of the disclosure.

As used herein, the articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Persons skilled in the art will understand that the elements and features shown or described in connection with one embodiment may be combined with those of another embodiment without departing from the scope of the present disclosure and will appreciate further features and advantages of the presently disclosed subject matter based on the description provided.

Throughout the present disclosure, terms such as “approximately,” “generally,” “substantially,” and the like should be understood to allow for variations in any numerical range or concept with which they are associated. For example, it is intended that the use of terms such as “approximately” and “generally” should be understood to encompass variations on the order of 25% (e.g., to allow for manufacturing tolerances and/or deviations in design).

Although the apparatus and methods of the subject disclosure have been described with respect to preferred embodiments, those skilled in the art will readily appreciate that changes and modifications may be made thereto without departing from the spirit and scope of the present disclosure.

What is claimed is:

1. A method of accelerating ingrowth of host living cells into a graft, the method comprising applying a mechanical tension over an interface of the graft and host cells to induce the ingrowth of host tissue.

2. The method of claim 1, wherein the graft has the fibrous structural framework of original tissue preserved.

3. The method of claim 1, wherein the graft is in the form of a paste or injectable fluid.

4. The method of claim 1, wherein the graft is derived from an allograft.

5. The method of claim 1, wherein the graft is derived from a xenograft.

6. The method of claim 1, wherein the graft is a material obtained from the patient.

7. The method of claim 1, wherein the mechanical tension is applied by a vacuum.

8. The method of claim 1, wherein an external expansion device is utilized to apply tension, and the device induces expansion by passively preventing intrinsic recoil of pre-stretched tissue.

9. The method of claim 1, wherein mechanical tension is applied by an external device applied sterile over a sterile graft matrix.

10. The method of claim 1, wherein mechanical tension is applied by a constant suction at a sufficient low level so as not to collapse capillary walls and block blood flow.

11. The method of claim 1, wherein mechanical tension is applied by an intermittent suction to provide relaxation periods to temporarily restore circulation.

12. The method of claim 1, further comprising infusing the graft matrix which one or more materials that invite tissue ingrowth.

13. The method of claim 12, wherein the one or more materials is infused prior to graft application and/or during graft application.

14. A method of augmenting soft tissue comprising injecting material derived from fat into a region of a body of a patient expanded by application of a distractive force to increase the volume of the region of the body.

15. The method of claim 14, wherein the graft is derived from an allograft.

16. The method of claim 14, wherein the graft is derived from a xenograft.

17. A method of augmenting soft tissue comprising injecting material derived from homologous graft into a region of a body of a patient expanded by application of a force to increase the volume of the region of the body.

18. The method of claim 17, wherein the injected material is derived from an animal.

19. The method of claim 17, wherein the injected material is derived from a human.

20. The method of claim 17, wherein the force induces a recipient site to generate a tissue engineering scaffold, the scaffold is seeded with an allograft or xenograft that has been processed to lose immunogenicity and maintain particular growth and differentiation factors and clues
